(19) United States
(12) Patent Application Publication
(10) Pub. No.: US 2009/0287302 A1
(43) Pub. Date: Nov. 19, 2009

(54) Polymer coated spinulose metal surfaces

(75) Inventors: Christina K. Thomas, Saint Paul, MN (US); Luke J. Ryves, Minneapolis, MN (US); Daniel M. Storey, Minneapolis, MN (US); Barbara S. Kitchell, Holmes Beach, FL (US)

Correspondence Address: CHAMELEON SCIENTIFIC CORPORATION;AKA IONIC FUSION CORPORATION, 13355 10TH AVENUE NORTH, SUITE 108, PLYMOUTH, MN, 55441, US

(73) Assignee: Chameleon Scientific Corporation

(21) Appl. No.: 12/152,698

(22) Filed: May 16, 2008

(51) Int. Cl. A61F 2/82; B32B 15/04; A61M 25/00

(52) U.S. Cl. 623/1.46; 427/576; 428/457; 428/469; 604/523

(57) ABSTRACT

Spinulose surfaces such as titanium and zirconium can be coated with a range of polymers used to form thin, adherent polymer surface films. Selected polymer coatings are useful for use as biocompatible surfaces on implants, catheters, guidewires, stents and a variety of medical devices for in vivo applications. The polymer coatings can also be used to protect metal surfaces nanostructured with spinulose titanium or zirconium.

### BACKGROUND OF THE INVENTION

1. Field of the Invention

The invention relates to polymer coatings and films and particularly to substrate surfaces coated with highly adherent thin polymer films on titanium or zirconium spinulose nanostructured substrates.

2. Description of Background Art

Polymer coatings on metals are useful in several applications, ranging from corrosion-inhibiting surfaces to biocompatible thin films on medical devices. Polymers with low coefficients of friction are desirable in catheters and guidewires used in surgical procedures and in permanently implanted devices such as stents and valves. Corrosion is a persistent problem with metals exposed to air and water; for example, the harsh environments encountered by steel rebars used in highways and bridges has led to increased use of deicing salts, which has accelerated corrosion damage.

Metals are used in the fabrication of several types of implants; however, bare metals used in stents, for example, provide a focus for restenosis, due to neointimal proliferation subsequent to implantation. Polymer coated stents have, in some instances, appeared to reduce the potential for the inflammation and thrombogenic reactions leading to restenosis. Many polymers are not suitable for implanted devices because of flexing or expansion upon implantation, in addition to peeling, cracking or detachment from the underlying metal substrate.

Several different types of polymers have been described as having properties useful for medical device coatings, ranging from polymers covalently attached to a metal surface to thin hydrogel films and biodegradable coatings.

Biocompatibility of the coating polymers is important. Billinger, et al. ((2006) reported decreased inflammation from poly(L-lysine)-graft-(polyethylene)glycol (PLL-g-PEG) coating which appears to reduce cell-stent interactions.

Ultraviolet light has been used to photocrosslink a biocompatible coating material associated with appropriate photoactive linking groups on a medical device causing the polymer to be covalently bound to the surface. Hergenrother, et al. in U.S. Pat. No. 5,750,206 describe coating hydrocarbon plasma treated metal surfaces with a crosslinkable polymer containing a latent photoactive chemical group that upon activation binds with the hydrocarbon treated surface. The coatings are described as lubricious and said to be suitable for guidewires.

As described in WO/1995/004839, pretreating metal guidewires with a hydrocarbon plasma deposits a residue over the metal, which acts as a tie layer for a subsequently applied outer hydrophilic polymer coating.

Other “layering” techniques have been used to prepare polymer-coated metal surfaces. U.S. Pat. No. 6,235,361 describes a metal surface coated with a thermoplastic polymer which has a peel strength at 130° C. An epoxy resin and a polypropylene binder are placed between the metal surface and a thermoplastic layer.

Polymer films have been textured to provide enhanced adhesion of plasma deposited metals. The morphology of the polymer surface is characterized by mounds and dimples, but the adherence of the polymer to an underlying surface is not addressed and the polymer structured surface is dependent on regulation of polymer phase kinetics (U.S. Pat. No. 6,099,939).

Many polymer coatings are not satisfactory for all types of surfaces, particularly for metal surfaces where a coating could provide protection from oxidative processes or increase or add desirable properties such as lubricity. The sloughing and peeling encountered with some polymer coated metal surfaces shows a lack of strong surface adherence to the substrate. This is of particular concern and interest in the development of biocompatible coatings on medical implants and other medical devices because the biocompatible properties of certain classes of polymers make them otherwise ideal for use on implants and other types of devices used in vivo.

### SUMMARY OF THE INVENTION

The present invention addresses the often troublesome sloughing and peeling of polymers used to coat and protect surfaces, particularly the biocompatible polymers currently used to coat surfaces of medical devices and to provide time release surfaces or matrices for various drugs.

During efforts to develop an effective control release coating over Ag/AgO, several PLLA films were coated onto the Ag/AgO previously vapor phase deposited on a conventional titanium surface. In all tests, the polymer coating sloughed from the smooth metal surface. The Ag/AgO was then vapor phase deposited onto a highly nanostructured titanium surface, selecting a spinulose titanium surface. The Ag/AgO adhered well to the surface, although the effect of a polymer coating over the Ag/AgO was not necessarily expected to act as a suitable controlled release coating. In fact, it was not clear that a polymer would adhere to the titanium spinules and/or the deposited Ag/AgO.

On both counts, the polymers tested showed that the spinulose titanium surface provided a strong attachment for the polymer and could effectively coat deposited Ag/AgO such that for appropriate polymers, a controlled release of silver could be achieved.

Accordingly, one embodiment of the invention is a polymer coated titanium or zirconium spinulose surface. Titanium or zirconium spinulose surfaces or films can be prepared on any type of substrate whether metal, polymer, glass, or ceramic The spinulose nanostructured substrate surfaces produced by a modified plasma deposition method shows that under certain controlled deposition conditions, a unique “spikey” metal film or coating can be produced on virtually any substrate (U.S. application publication No. ______). The present invention demonstrates that such spikey surfaces generated from titanium or zirconium are surprisingly well suited for top coating with a wide range of polymers. Appropriate polymers can be selected as required for specialized utilities such as protective coatings, anchors or matrices, and controlled elution coatings.

Titanium spinulose surfaces on a metal, polymer, ceramic or glass substrate surface are highly nanostructured, but maintain basic structure when coated with Ag/AgO or thin layers of drugs/biomolecules, see FIG. 2.

In practicing the invention, a substrate surface is first modified with nano plasma deposited (NPD) spinulose titanium nanoparticulates, followed by application of the polymer onto the nanoparticulate surface. Depending on the polymer, the application may be by casting, spraying, dipping, electrospinning, or similar methods. In some applications, it may be advantageous to apply a polymer by vapor deposition, such as a plasma-enhanced chemical vapor deposition. Some monomers may polymerize on the spinulose surface and can be employed to form very thin films.

Using the procedures described herein, polymers are durably attached to surfaces that would otherwise exhibit only weak or unpredictable attachment properties. The thickness of films can be controlled by the deposition method; for example, several dipping steps after initial dipping or formation of a polymer layer on the spinulose titanium surface can be used to provide thicknesses varying up to several microns.

The unique structure of the spinulose surface is produced by controlled nanoplasma deposition. As discussed, a polymer can be dispersed on this surface also using a vapor deposition method, but in some cases more conveniently by simple dipping. It is believed that many agents, including bioactive materials such as therapeutic drugs, can be effectively co-deposited or serially deposited with the polymer. When co-deposited with a polymer and depending on the polymer, the agent can be released or eluted from the polymer matrix in a time-dependent manner. Different time release profiles can be developed for agents deposited in combination with a coating polymer.

Accordingly, the invention provides a method to efficiently attach polymers to a uniquely spinulose substrate surface, not only providing excellent adhesion and durability, but also avoiding complicated, hazardous and inefficient chemistry; e.g., the silane, photo-, thermo-couplings used for polymer attachment, as well as ultraviolet and heating steps that may cause surface damage. An additional advantage of the invention is the option to use polymers with functional groups, in effect providing an additional functional feature to the surface without employing additional steps to modify the deposited polymer.

Nanostructured spinulose metal surfaces act as scaffolds for polymer surfacing and for molecules initially deposited onto such a nanostructured surface. In preferred embodiments, biomolecules and/or bioactive agents, including metals such as silver, are deposited on the spinulose surface by nano or molecular plasma deposition, or by other conventional and well-know deposition methods, such that the nanostructure of the spinulose surface is preserved. In the example of Ag/AgO nanoplasma deposition on a spinulose titanium surface, the SEM photograph as seen in FIG. 2, indicates that the titanium spikes appear coated but otherwise retain similar nanorough structure. The general nanoroughness is not lost as can be seen by comparison with the SEM photograph in FIG. 3 of uncoated spinulose titanium.

The polymer films applied on metal spinulose surfaces are extremely resistant to shear and thermal peeling. Depending on the polymer, the preparation can be rapid and cost-effective.

An advantage of preparing polymer surface films on spinulose metal surfaces is that many types of polymers can be applied to such surfaces by any of a number of application methods. A preferred method applicable to several types of polymers is a simple dipping procedure, which is rapid and inexpensive compared to other surface coating methods, including spraying, casting, spin coating or plasma deposition.

Several types of polymers can be polymerized on the spinulose metal surface, including thermosetting polymers, polymerized from monomers requiring either low or high polymerization temperatures. A spinulose surface, for example, can be contacted with either low or high polymerization temperatures as required for many thermosetting polymers. High polymerization temperatures can be employed without significant changes to a spinulose metal surface, for example, in view of titanium's melting temperature of over 1000° C. Photopolymerizable molecules requiring use of ultraviolet light or other radiation also would not affect the underlying spinulose metal surface. A wide range of polymers are suitable for coating on spinulose metal surfaces. Thus a significant advantage of the spinulose metal surfaces is that surface structure and binding properties can be maintained even if heating is required to cure or polymerize a precursor monomer.

There are several advantages to polymer films that are strongly and durably adhered to surfaces with spinulose metal surface features. Biodegradable, biocompatible polymers can serve as a diffusion barrier against a reservoir device; e.g., silver oxide, to control release rate. A semi-permeable membrane over a drug-loaded surface with select polymer/copolymers can be fabricated to meet specific functional requirements. Similarly, a drug can be loaded onto a spinulose metal surface and used to create a controllable drug delivery system with a biodegradable polymer(s)/co-polymer(s) for controlled release. Alternatively, a bioactive agent can be dispersed or dissolved in an inert polymer that is then cast or sprayed on a spinulose metal surface.

Functional polymers can also be used. Examples include monofunctional or bifunctional thiol, amino, maleimidyl, p-nitrophenyl, carboxyl, aldhyde active and/or N-hydroxysuccimidyl activated ester PEG polymers or any polymer derivative, and the like, adhered to a spinulous surface which can serve as a platform for attachment of biological molecules. Depending on the choice of polymer, one can introduce other desirable characteristics to the substrate surface. Examples include conjugation of biomolecules to the active sites of a dicarboxylic acid-PEG while simultaneously utilizing the PEG chain of the same molecule for protein passivation; improving cell adhesion by introducing not only an underlying nanostructured surface, but also a nanostructured surface topically modified with a biological polymer, such as collagen fibronectin, vitronectin, laminin and the like.

Nanotextured spinulose metal surfaces can be produced by controlled nanoplasma deposition (NPD) of titanium and/or zirconium on a wide range of substrate surfaces. Nano plasma deposited titanium and/or zirconium exhibits features significantly different in appearance from most other vapor deposited metals and metal compounds. The nano-rough surface appears during the deposition as spikes on round particulates when the deposition is cycled under certain controlled conditions.

The deposition process that produces a spinulose surface is a modified ion plasma deposition process in which a plasma is generated from metal target and deposited onto a substrate under reduced pressure. The metal plasma deposits as nanoparticulates, atoms and ions, which after further deposition under the described controlled deposition cycling conditions will form unusual nanostructured surfaces.

### DEFINITIONS

Surfaces having a spiney appearance are characterized as “spinulose” as defined in Random House Unabridged Dictionary. Spinules are distinguished in appearance from larger, more hair-like appendages commonly characterized as whiskers or columnar structures and which are typically wire or rod-like in appearance.

Spinulose metal surfaces are produced under special nanoplasma deposition conditions. The surfaces are unique in appearance, showing distinctly pointed spikey projections over the surfaces.

As used herein, “substantially” is intended to indicate a limited range of up to 10% of any value indicated.

As used within the context of the claimed subject matter, the term “a” is not intended to be limited to a single material or element.

Physical vapor deposition (PVD) is used to describe a class of processes that involve the deposition of material, often in the form of a thin film, from a condensable vapor which has been produced from a solid precursor by physical means. There are many ways of producing the vapor, and many modifications to each of these processes. Examples of PVD processes include evaporation, sputtering, laser ablation and arc discharge. PVD can involve chemical reactions, such as from multiple sources, or by addition of a reactive gas.

Electron beam evaporation is use of an electron beam to heat a metal so that it evaporates. The vapor can be deposited on a surface.

### BRIEF DESCRIPTION OF THE FIGURES

FIG. 1 is a sketch of a typical ion plasma deposition apparatus; pure metal cathode target 1; substrate 2; substrate holder 3; vacuum chamber 4; power supply for target 5; and arc control 6. Not shown is an inlet into the vacuum chamber 4 for introducing a gas flow, which may be an inert gas, or reactive gas such as oxygen.

FIG. 2 is a SEM image of Ag/AgO deposited by nanoplasma deposition onto a spinulose titanium surface on a titanium substrate.

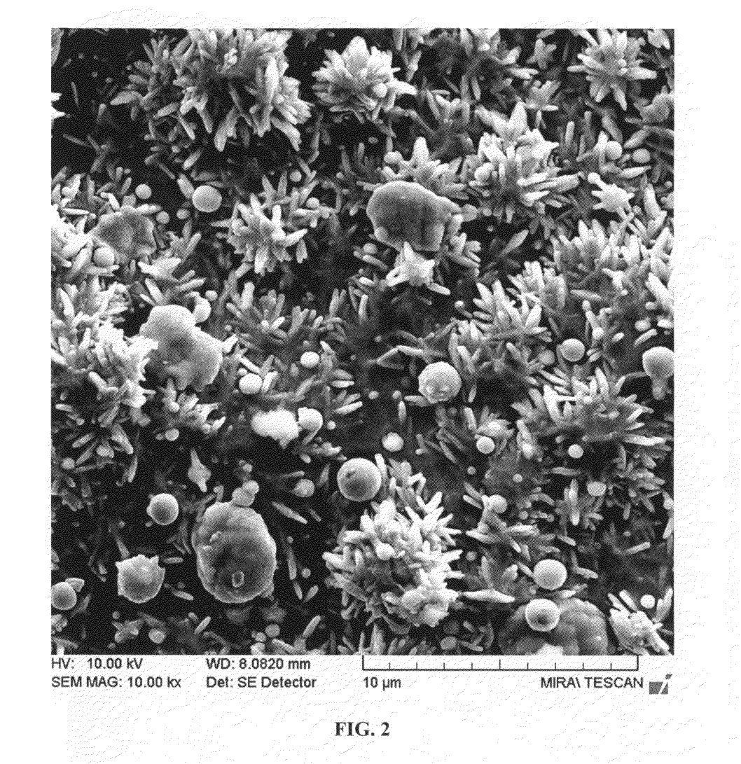

FIG. 3 is an SEM image of a titanium spinulose coating formed from a titanium plasma deposited on a titanium substrate.

FIG. 4 is an SEM image of a zirconium spinulose coating formed from a zirconium plasma on a titanium substrate.

FIG. 5 is an SEM image of PLLA coated spinulose titanium scratched with a conospherical scratch probe with increasing normal load.

FIG. 6 is an SEM image of PLLA coated on smooth titanium scratched with a conospherical scratch probe with increasing normal load.

FIG. 7 is an elution profile of silver from Ag/AgO deposited on a spinulose titanium surface without a PLLA polymer coating (o) compared with silver eluted from Ag/AgO coated on a spinulose titanium surface with PLLA polymer coating (x). Elutions were performed in phosphate buffered saline (1× PBS) and mL/cm² [Ag] measured by ICP.

FIG. 8 is a photograph image of PLLA coated spinulose titanium nanostructured substrate following a tape test.

FIG. 9 is a photograph image of PLLA coated smooth titanium substrate following a tape test.

### DETAILED DESCRIPTION OF THE INVENTION

In order to prepare surfaces for attaching polymer coatings, conventional texturing techniques such as sandblasting have often been used by others to improve polymer adherence. Yet lack of polymer adherence remains a concern. The present invention utilizes a new nanotexturing technique that creates a nanostructured surface on a substrate in the form of spinulose nanoparticulates. These surfaces are distinctly different from whiskered type metal surfaces or from the columnar type of thin film surfaces described by Robbie and Brett, (1997) obtained by using a plasma vapor deposition. The nanostructured metal surfaces are also distinct from the intergranular etched polymer surfaces to which an immersion plated metal is applied leading to increased peel strength (U.S. Pat. No. 6,506,314).

This unique spikey surface has been grown on several metal, polymer, ceramic and glass substrates from titanium or zirconium using a modified nanoplasma deposition process.

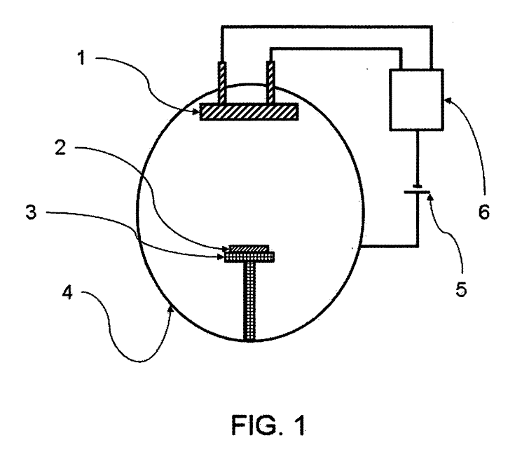

The apparatus for plasma deposition of these metals is shown in FIG. 1. The deposition process, a modified plasma depostion as described herein, provides uniquely nanotextured spinulose metal surfaces which can be used as surfaces for strong attachment of polymers. Polymer surfaces can retain surface nanofeatures and offer an additional platform for incorporating dual functionality onto substrate surfaces, as attachments to the polymer itself or as overlying protective coatings.

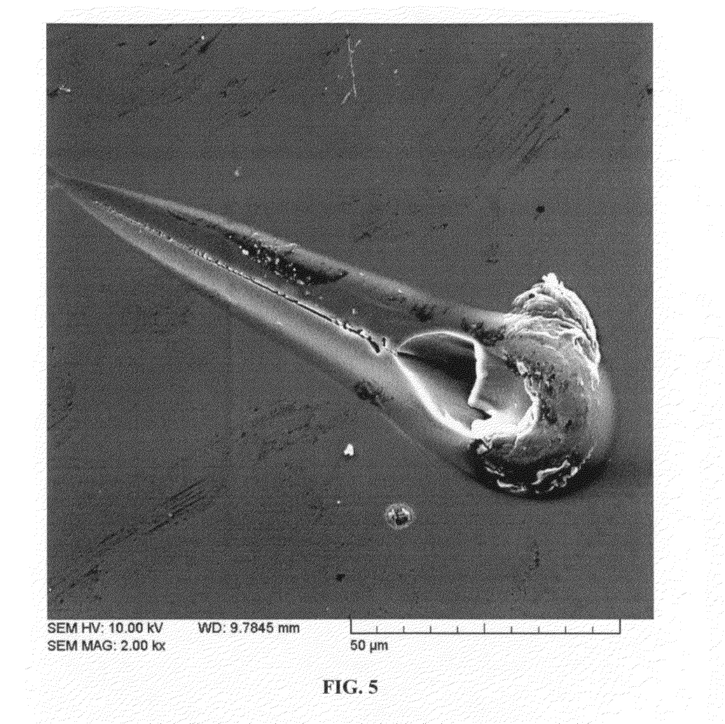

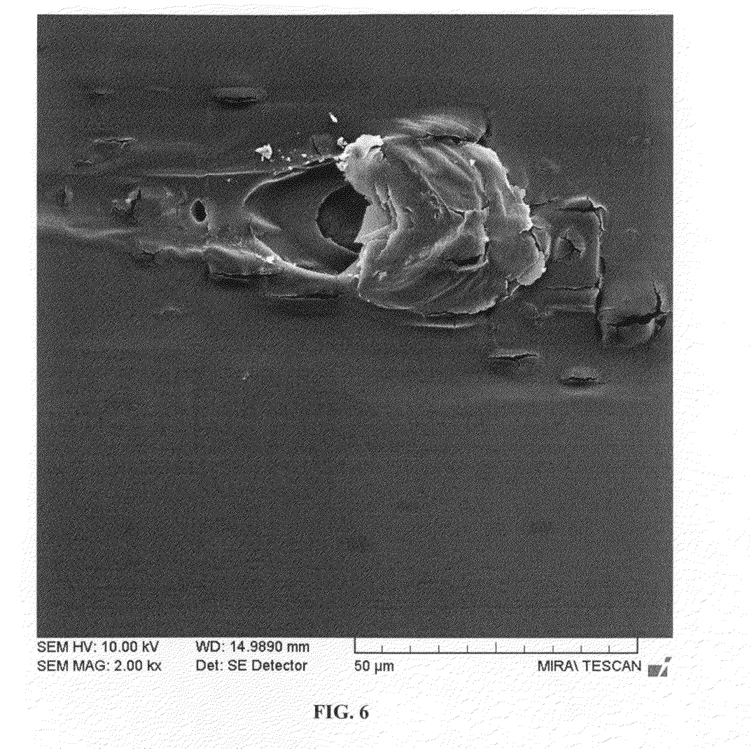

Metal surface features contribute to the reaction of metals with external environments and in the determination of binding properties with other materials. Accordingly, the ability to produce adherent polymer coatings and films on spinulose surfaces makes it possible to protect a metal substrate from external forces and/or to endow a substrate surface with functional or linking groups suitable for attaching biomolecules such as drugs. Polymers of many different types are suitable for applying to a nanoplasma deposited spinulose nanoparticulate surface, including hydrophilic, hydrophobic and functionalized polymers. FIG. 5 illustrates the strong adherence of PLLA coated spinulose titanium (FIG. 5) compared to the poor adhesion of PLLA coated over smooth titanium (FIG. 6).

Polymers may be applied to the spinulose surfaces by any of several convenient coating methods, including dipping, spin coating, spraying, flood coating or the like. Plasma deposition methods may also be used.

Additionally, polymer coatings may act as time release barriers for selected bioactive agents, particularly those used in conjunction with medical device coatings. In an illustrative example, poly-L-lactic acid (PLLA) was tested because of its biocompatibity and potential application for coatings on stents, guidewires and various implants. PLLA and poly(lactic-co-glycolic acid) (PLGA) coatings were applied as diffusion barriers over reservoirs of Ag/AgO deposited on spinulose titanium substrates. Silver released from surface-deposited Ag/AgO has recognized antimicrobial properties and has been used as an antimicrobial agent externally and as a coating on in vivo devices.

The polymer coatings on Ag/AgO deposited onto a titanium spinulose surface demonstrated that PLLA and PLGA could sustain the release of silver over at least several days, while simultaneously maintaining polymer integrity on the surface. This demonstrated that selected polymer coatings over bioactive agents and/or biomolecules deposited on spinulose titanium surfaces do not peel or slough from the surface and, importantly, can be used for timed or controlled release. While illustrated with Ag/AgO release, it is expected that drugs, including a wide range of organic molecules, as well as compounds that are metallic or include metals, can be attached or deposited onto a spinulose surface, coated with a suitable polymer and further developed for a desired time release profile.

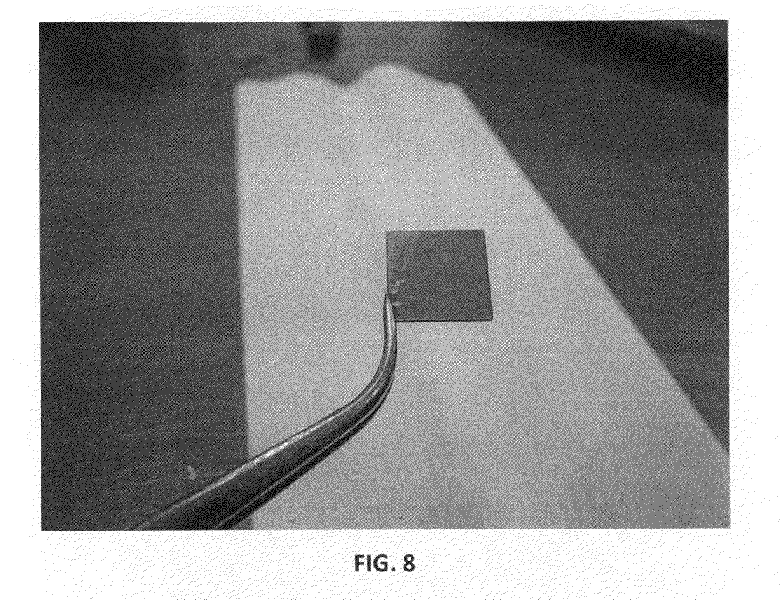

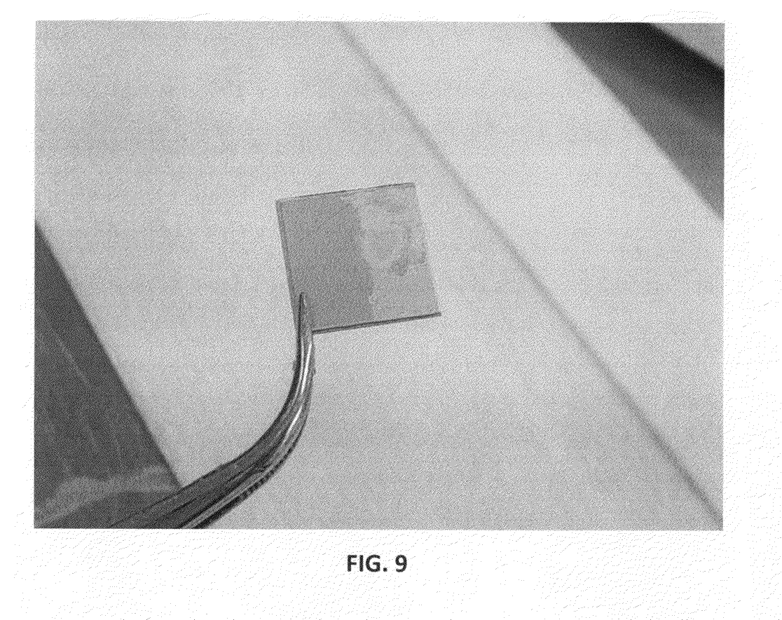

Tape tests confirmed that the adherence of polystyrene (PS), poly(lactic-co-glycolic acid)(PLGA), poly-L-lactic acid (PLLA) and polyethylene glycol (PEG) polymers to spinulose nanostructured titanium surfaces was surprisingly high and significantly better than adherence to smooth titanium surfaces. PS, PLGA, PLLA and PEG coatings were applied to spinulose titanium substrates as well as to smooth titanium surfaces in order to compare adhesion. Adhesion was determined by using a tape test as described in ASTM D3359-08. This standard practice demonstrated that polymer coatings with a range of chemical properties, tightly adhered to spinulose nanostructured surfaces but failed to remain completely intact on a smooth titanium surface as shown in FIG. 8 and FIG. 9, respectively.

FIG. 5 demonstrates the enhanced adhesive properties of PLLA to a spinulose nanostructured titanium surface when using a conspherical scratch probe with increasing load normal to test the interfacial adhesion of PLLA to the spinulose nanostructured titanium substrate. The PLLA coating displayed good adhesion even around the severely damaged areas. In contrast, the same test with PLLA coated smooth titanium resulted in delamination of a region around the load, causing buckling and cracking of the polymer film, as illustrated in FIG. 6.

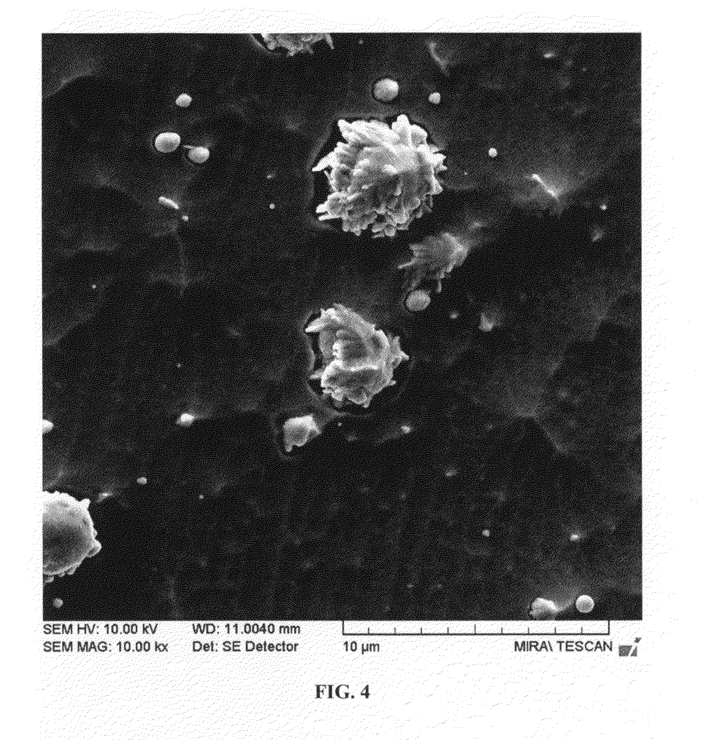

Spinulose titanium nanostructured surfaces can be produced with commercially pure titanium (grade 2) and with zirconium. Spinulose surfaces, using conditions described for producing titanium spinulose surfaces, were obtained with zirconium, are shown in FIG. 4.

The spinulose-type surfaces produced from titanium and zirconium under the described conditions have not been observed with aluminum, cobalt, copper, nickel, hafnium, 316L stainless steel, nitinol, silver or titanium 6-4 metal targets deposited on stainless steel substrates. On the other hand, in some cases, these metals form other types of unusual nanostructured surfaces which are distinctly different from the spinulose appearance of deposited titanium and/or zirconium. Generally, with the exception of aluminum, the nickel, cobalt, copper, silver, hafnium, 316L stainless steel, nitinol and titanium 6-4, nanostructured surfaces are basically globular or stacked globular in shape. Aluminum was distinctly different from titanium and the other metals cyclically deposited NPD metals.

Pure aluminum metal deposited under the same conditions described for titanium and/or zirconium has a stacked appearance with a geometric cube-like structure different from the structures observed with titanium and other metals. While spinulose surfaces for aluminum and other metals are not observed under the conditions used to produce spinulose titanium or zirconium nanostructured surfaces, it may be possible to generate spinules by using modifications of the disclosed deposition procedures, such as, but not necessarily limited to, longer intervals between deposition cycles, distance from target and chamber pressure.

Spinulose nanostructured titanium and zirconium surfaces can be formed as coatings or films on virtually any metal, plastic, ceramic or glass substrate surface, including stainless steel, titanium, CoCrMo, nitinol, glass or silicon, as well as on silicone, poly(methylmethacrylate) (PMMA), polyurethane (PU), polyvinyl chloride (PVC), polyethylene terephthalate glycol (PETG), polyetheretherketone (PEEK), polytetrafluoroethylene (PTFE), polyethylene terephthalate (PET), ultra high molecular weight polyethylene (UHMWPE), and polypropylene (PP). Other metals, including aluminum, gold, platinum, copper and silver are also suitable substrates.

### EXAMPLES

### Example 1

### Spinulose Titanium or Zirconium

Nanostructured spinulose titanium or zirconium surfaces can be produced by a modified cyclic plasma arc deposition procedure termed nano plasma deposition (NPD). The apparatus for producing the metal ion plasmas is shown in FIG. 1.

The selected substrate material was ultrasonically cleaned before deposition in detergent (ChemCrest #275 at 160° F.), rinsed in deionized water and dried in hot air.

The clean substrate was then placed in the chamber and exposed to nano-plasma deposition (NPD) using the special deposition conditions described. The cathode was commercially pure titanium cathode (grade 2) or zirconium 7021. The substrates were mounted in the vacuum chamber at distances from 6 to 28 in from the cathode (measured from the centre of the cathode). The angle between the cathode surface normal and a line from the centre of the cathode to the substrate, θc, was varied in the range 0-80°. The angle between the depositing flux and the substrate surface normal, θs, was varied in the range of 0-80°.

The angle between the substrate surface normal and a line from the centre of the cathode to the substrate, θc, was varied in the range of 0-80°. The angle between the depositing flux and the substrate surface, θs, was varied in the range of 0-80°. The chamber was pumped to a base pressure of between 1.33 mPa and 0.080 mPa. The arc current was varied from a 15-400 A with an argon burn pressure of 0.1 to 5.5 mT.

The process was run in cycles, with each cycle consisting of plasma discharge intervals (varied over the range 1 to 20 minutes) followed by intervals where there was no discharge and no gas flow (between 5 and 810 minutes). Each process consisted of 3-27 cycles.

The apparatus for the plasma deposition is shown in FIG. 1. The metal cathode targets are disposed in a vacuum chamber. An inert gas, typically argon, is not required but may be introduced into the evacuated chamber and deposition commenced. The substrate 2 is generally positioned 6-28 inches from the target and deposition is conducted intermittently for periods of approximately 1-20 minutes. During the intervals between depositions, there is no plasma discharge and the inert gas flow optionally can be reduced or stopped completely if desired. The intervals between depositions can be varied and are about 5-90 min with a typical run of about 3-27 cycles.

Following plasma deposition, the samples were characterized by scanning electron microscopy (SEM). SEM images were obtained with a Tescan Mira Field Emission instrument (Brno, Czech Republic, Jihomoravsky, Kray) equipped with a SE detector, at a magnification of 50 K and 10 K times at 10 kV.

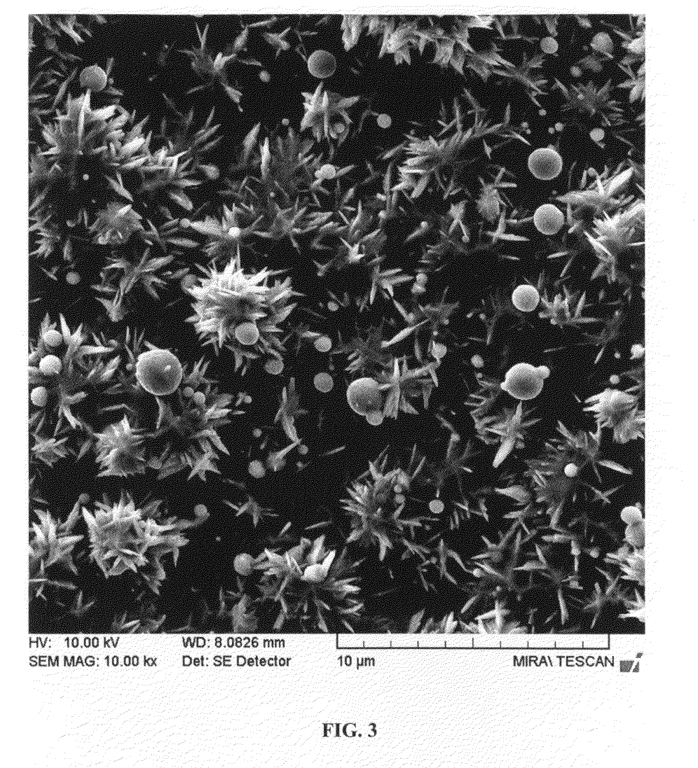

Initially NPD deposited particles are typically round and will differ in size and distribution depending on power and/or time of deposition. Under the described specified deposition conditions, titanium or zirconium metal particles develop nanosized spike-like protrusions, which were observed as spinules or small thorny spines as shown in FIG. 3 or FIG. 4, respectivley.

### Example 2

### Nano Plasma Deposition of Silver/Silver Oxide

Ionic Plasma Deposition (IPD), similar to the process for NPD, creates a highly energized plasma from a target material, typically solid metal, from a cathodic arc discharge. An arc is struck on the metal and the high power density on the arc vaporizes and ionizes the metal, resulting in a plasma which sustains the arc because the metal vapor itself is ionized, rather than an ambient gas.

An apparatus suitable for controlling deposition of a silver/silver oxide plasma ejected from a silver cathodic arc target source 1 onto a substrate 2 is shown in FIG. 1 within the vacuum chamber 4 or by a power supply 5 to the target and adjustment of arc speed 6. The closer a substrate is to the arc source, the larger and more densely packed will be the particles deposited on the substrate.

A 4% w/v poly-L-lactic acid polymer solution in chloroform was cast over the surface of a Ag/AgO coated smooth titanium substrate from a pipette. The polymerized coating was only weakly adherent to the underlying silver surface as evidenced by peeling of the film shortly after immersion in phosphate buffered saline (PBS) or deionized water at 37° C. in less than one day.

### Example 4

### Polymer Film on Ag/AgO Coated Spinulose Titanium

A 4% w/v poly-L-lactic acid polymer solution in chloroform was cast from a pipette over Ag/AgO deposited onto a spinulose titanium surface. The polymer coating was strongly adherent to the underlying silver spinulose surface and was not easily peeled from the surface. Adhesion was tested as described in Example 5.

### Example 5

### Polymer Adhesion to Spinulose Titanium Surfaces

Interfacial adhesion of PS, PLGA, PLLA and PEG coatings to spinulose titanium and to smooth titanium surfaces were compared using a scratch induced delamination process. This test demonstrated that the polymer coatings, with a range of chemical properties, exhibited little, if any, delamination from the spinulose nanostructured titanium surface, while the polymers were typically observed to fracture and in many cases fall off the smooth titanium surface. FIG. 5 shows the enhanced interfacial adhesion properties of PLLA to a spinulose nanostructured titanium surface following a scratch test compared to the poor adhesion properties of PLLA to the smooth titanium, FIG. 6. The work done in both of these scratch tests was similar. The lack of delamination evident from observations with light microscopy showed that the interface is considerably toughened with the spinulose surface. The scanning electron microscopy (SEM) revealed a difference in failure modes, shown in FIG. 6, with the non-spinulose sample showing cracks in the polymer coating above regions subject to delamination that were not observed in the spinulose coated sample, FIG. 5.

### Example 6

### Elution of Silver from PLLA Coated Ag/AgO on Spinulose Titanium

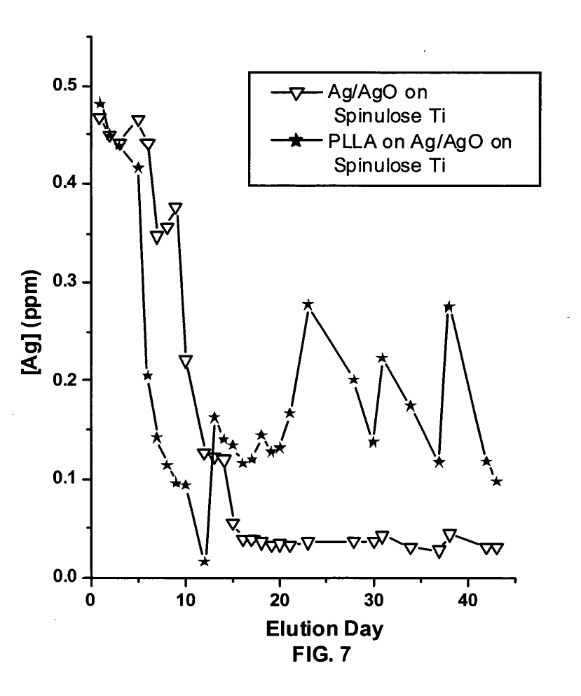

A spinulose titanium surface was formed on a smooth titanium substrate as described in Example 1. Ag/AgO was deposited on the spinulose surface by ion plasma deposition (IPD) from a silver cathode as described in Example 1 with use of a silver target. A film of PLLA was then cast over the Ag/AgO as described in Example 4. The coated Ag/AgO was placed in deionized water, physiological saline or PBS at 37° C. FIG. 7 shows an elution profile in PBS for silver after 43 days comparing silver profiles of PLLA coated Ag/AgO and uncoated Ag/AgO deposited on a spinulose titanium surface. At day 13 in the PBS, the Ag/AgO remaining on the PLLA coated spinulose titanium surface was higher than the amount deposited on the Ag/AgO spinulose titanium only surface. Even after soaking for at least 43 days in deionized water, the polymer film remained well adhered to the spinulose surface.

### REFERENCES

WO/1995/004839

U.S. Pat. No. 6,235,361

U.S. Pat. No. 5,750,206

Billinger, M., et al. “Polymer Stent Coating for Prevention of Neointimal Hyperplasia” J. Invasive Cardiology, v 18(9), 423-426 (2006)

U.S. Pub. No. 2007/0071879

Kumar, V. R. and Fradeep, T., “Polymerization of benzylthiocyanate on silver nanoparticles and the formation of polymer coated nanoparticles” J. Mater. Chem., v 16, 837-841 (2006)

U.S. Pat. No. 6,725,878

U.S. Pat. No. 6,099,939

U.S. Pat. No. 6,063,314

U.S. Pub. No. 2007/0071879

U.S. Pat. No. 5,750,206

U.S. Pat. No. 6,506,314 (Jan. 14, 2003)

U.S. Pat. No. 6,099,939

www.invasivecardiology.com/article/6092

U.S. application Pub. No. ______ (pending unpub app)

1-21. (canceled)

22. A polymer or copolymer coated nanoplasma deposited titania or zirconium surface having nanosized spike-like thorny protrusions (spinulose) emanating radially from rounded surface deposited metal nanoparticles on a substrate.

23. The coated spinulose surface of claim 22 which exhibits enhanced surface adherence for the polymer or copolymer compared to a smooth titanium surface coated with said polymer of copolymer.

24. The coated surface surface of claim 22 comprising deposited spinulose titanium and zirconium.

25. The polymer coated surface of claim 22 which is on a titanium spinulose surface.

26. The polymer coated surface of claim 22 wherein the polymer is a biodegradable polymer or copolymer.

27. The polymer coated surface of claim 26 wherein the biodegradable polymer or copolymer is poly-L-lactic acid (PLLA), poly-lactic-co-glycolic acid) (PLGA) or a combination of PLLA and PLGA.

28. The polymer coated surface of claim 25 wherein the polymer is bound to a bioactive agent.

29. The polymer coated surface of claim 28 wherein the bioactive agent is an antimicrobial agent.

30. A medical device comprising a polymer coated nanorough titanium or zirconium spinulose surface characterized by round nanoparticulates with radially disposed nanosized spike-like projections.

31. The device of claim 30 wherein the spinulose surface is titanium.

32. The device of claim 30 wherein the device is a stent, guidewire, catheter or implant.

33. The device of claim 30 wherein the titanium or zirconium spinulose surface is on a metal, polymer or ceramic substrate.

34. The device of claim 30 wherein the polymer coating is a biodegradable polymer or copolymer.

35. The device of claim 30 wherein a bioactive agent is attached or adhered to the spinulose surface.

36. The device of claim 29 wherein the spinulose surface is nanodeposited titanium and zirconium.
